(19) United States
(12) Patent Application Publication
(10) Pub. No.: US 2004/0088014 A1
(43) Pub. Date: May 6, 2004

(54) Multi-site anti-tachycardia pacing with programmable delay period

(76) Inventor: John E. Burnes, Andover, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/284,875

(22) Filed: Oct. 31, 2002

(51) Int. Cl. A61N 1/362

(52) U.S. Cl. 607/14

(57) ABSTRACT

The invention provides techniques for delivering anti-tachycardia pacing therapies to a heart. A medical device for providing anti-tachycardia therapy consistent with the invention may include two or more electrodes located proximate to or within the ventricles and/or two or more electrodes located proximate to or within the atria of a heart for treating ventricular and/or atrial tachycardias. At least some of the pulses within a sequence of pulses of a selected therapy may be delivered via each of the two or more electrodes. The timing of the delivery of these pulses by a particular electrode may be based on a programmed cycle length between consecutive pulses within the sequence and delay periods that are programmed for each electrode for each of these pulses. Thus, different electrodes may deliver the same pulse within a sequence at different times, increasing the effectiveness of anti-tachycardia pacing therapies.

### TECHNICAL FIELD

The invention relates to cardiac therapy, and more specifically to methods and processes that may be employed by medical devices to terminate tachycardias of a heart.

### BACKGROUND

An arrhythmia is a disturbance in the normal rate, rhythm or conduction of the heartbeat. Arrhythmias may originate in the atria or ventricles. Atrial tachycardia (AT) and ventricular tachycardia (VT) (collectively referred to as tachycardias), are forms of arrhythmia in which the atria or ventricles contract at a high rate, e.g., 100 or more beats per minute. Atrial fibrillation (AF) and ventricular fibrillation (VF) (collectively referred to as fibrillation) are other forms of arrhythmias, characterized by a chaotic and turbulent activation of atrial or ventricle wall tissue. The number of depolarizations per minute during fibrillation can exceed 400.

Ventricular tachycardias can lead to loss of consciousness, and in some cases can be life threatening. Moreover, ventricular tachycardias can lead to ventricular fibrillation, which, if untreated, will lead to loss of consciousness within a matter of seconds and death within a matter of minutes. While atrial tachycardias are generally not life threatening, they may lead to heart failure, ventricular tachycardia, or ventricular fibrillation. Both ventricular and atrial tachycardias are also associated with other low cardiac output symptoms, such as fatigue, and if left untreated, can lead to other dangerous life-threatening conditions, such as the development of blood clots that can cause stroke and possibly death.

Treatment for atrial or ventricular tachycardias may include anti-tachycardia pacing (ATP), in which one or more trains of high rate pulses are delivered to the heart in an attempt to restore a more normal rhythm. ATP is typically effective in converting stable tachycardias to normal sinus rhythm, and is often delivered via an implantable medical device. In many cases, a sequence of increasingly aggressive ATP therapies is delivered until an episode of tachycardia is terminated. The implantable medical device can be configured to discontinue ATP and immediately deliver a cardioversion or defibrillation shock to the heart in the event the tachycardia degrades into fibrillation.

For some tachycardia episodes, existing ATP techniques may not be effective. A tachycardia episode may originate in a very localized site within a specific heart chamber. It is believed that ATP terminates a tachycardia episode through the interactions between the depolarization wave fronts caused by the pacing pulses and the depolarization wave front of the tachycardia. Existing ATP techniques may deliver the pacing pulses at locations or times such that these interactions are not effective to end a particular tachycardia.

### SUMMARY

In general, the invention is directed to methods and processes for delivering anti-tachycardia pacing therapies to a heart. An implantable medical device, for example, for providing anti-tachycardia therapy consistent with the invention may include two or more electrodes located proximate to or within the ventricles and/or two or more electrodes located proximate to or within the atria of a heart for treating ventricular and/or atrial tachycardias. At least some of the pulses within a sequence of pulses of a selected therapy may be delivered via each of the two or more electrodes. The timing of the delivery of these pulses by a particular electrode may be based on a programmed cycle length between consecutive pulses within the sequence and delay periods that are programmed for each electrode for these pulses. Thus, different electrodes may deliver the same pulse within a sequence at different times, increasing the effectiveness of anti-tachycardia pacing therapies.

The implantable medical device may also classify detected tachycardias and associate classified tachycardias with therapies that are successful and unsuccessful in terminating the classified tachycardias. Successful therapies may be applied to later detected tachycardias that are similar to previously classified tachycardias, and unsuccessful therapies may be avoided when selecting therapies to treat later detected tachycardias that are similar to previously classified tachycardias. Classification of tachycardias may further improve the effectiveness of the anti-tachycardia pacing therapies.

In one embodiment, the invention is directed to a method that includes selecting an anti-tachycardia pacing therapy that includes at least one sequence of pulses, and delivering at least some of the pulses of at least one sequence to the heart via each of at least two electrodes based on programmed cycle lengths between consecutive pulses of the sequence and delay periods that are programmed for each of the electrodes. The anti-tachycardia pacing therapy may be selected in response to detection of a tachycardia of the heart.

In another embodiment, the invention is directed to a device that includes at least two electrodes and a control unit. The electrodes deliver pacing pulses to the heart. The control unit selects an anti-tachycardia pacing therapy that includes at least one sequence of pulses, and directs output circuits associated with the electrodes to deliver at least some of the pulses of at least one sequence to the heart via each of the electrodes based on programmed cycle lengths between consecutive pulses of the sequence and delay periods that are programmed for each of the electrodes. The electrodes may sense electrical activity within the heart, and the control unit may detect a tachycardia of the heart based on the electrical activity and select the therapy based on the detection.

In another embodiment, the invention is directed to a computer-readable medium containing instructions. The instructions cause a programmable processor to select an anti-tachycardia pacing therapy that includes at least one sequence of pulses, and deliver at least some of the pulses of at least one sequence to the heart via each of at least two electrodes based on programmed cycle lengths between consecutive pulses of the sequence and delay periods that are programmed for each of the electrodes. The medium may further contain instructions that cause a processor to detect a tachycardia of a heart, and select the therapy in response to the detection.

In another embodiment, the invention is direct to a method that includes detecting a tachycardia of a heart with a medical device, automatically selecting an anti-tachycardia pacing therapy that includes at least one sequence of pulses in response to the detection, delivering a pulse within the sequence to the heart via a first electrode at a first time, and delivering the pulse to the heart via a second electrode at a second time that is subsequent to the first time. The second time may be a programmed delay period associated with the second electrode subsequent to the first time.

The invention may be capable of providing a number of advantages. For example, providing anti-tachycardia pacing pulses via two or more electrodes increases the likelihood that the stimulation will be near the site of origination of the detected tachycardia. Further, providing a programmed delay period between the delivery via the electrodes for some pulses or sequences may alter the interactions of the depolarization wavefronts caused by the pulses and the wavefront caused by the tachycardia. These advantages in turn may increase the likelihood of capturing the myocardial tissue ahead of the depolarization wave front caused by the tachycardia, increasing the effectiveness of tachycardia therapy.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic view of an exemplary implantable medical device within a human patient.

FIG. 2 is another schematic view the implantable medical device of FIG. 1 located in and near a heart.

FIG. 3 is a functional block diagram of the implantable medical device of FIGS. 1 and 2.

FIGS. 4A-D are timing diagrams illustrating the delivery of anti-tachycardia pacing pulses by an implantable medical device according to the invention.

FIG. 5 is a flow chart illustrating an exemplary method for delivery of anti-tachycardia pacing therapy.

FIG. 6A and 6B are flow charts illustrating an exemplary method for classifying tachycardias and selecting anti-tachycardia pacing therapies.

### DETAILED DESCRIPTION

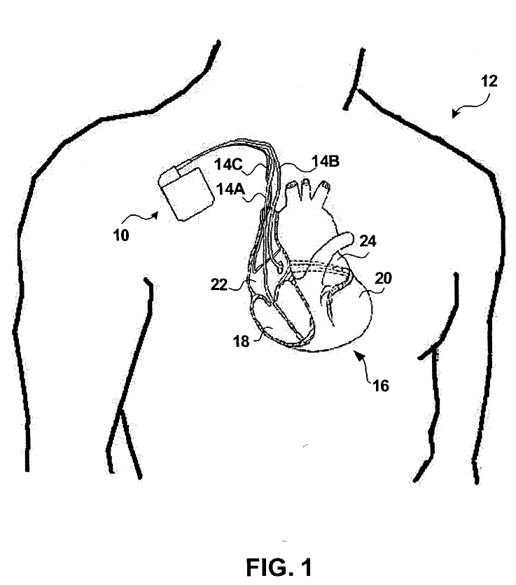

FIG. 1 is a schematic view of an exemplary implantable medical device (IMD) 10 implanted within a patient 12. IMD 10 may be a pacemaker, and in some embodiments, may be a pacemaker-cardioverter-defibrillator (PCD). IMD 10 includes at least two sensing and pacing leads 14A and 14B (collectively “leads 14”) that sense electrical signals attendant to the depolarization and repolarization of heart 16, and further provide pacing pulses for causing depolarization of cardiac tissue in the vicinity of the distal ends thereof. As shown in FIG. 1, the distal ends of leads 14A and 14B may be located within the right ventricle 18 and proximate to the left ventricle 20 of heart 16, respectively. IMD 10 may include any number of additional sensing and pacing leads 14, such as sensing and pacing lead 14C whose distal end is shown in FIG. 1 as located within right atrium 22. Leads 14 may have unipolar or bipolar electrodes disposed thereon, as is well known in the art.

IMD 10 is not limited to the configuration associated with leads 14 illustrated in FIG. 1. In some embodiments, IMD 10 includes at least one lead 14 located within or proximate to each of ventricles 18 and 20. In some embodiments, IMD 10 includes at least one lead 14 located within or proximate each of atria 22 and 24. In some embodiments, IMD 10 includes two or more leads 14 within or proximate to any one of chambers 18-24. In other words, leads 14 of IMD 10 may be configured in any way such that at least two leads 14 are located within or proximate to ventricles 18,20, or at least two leads are located within or proximate to atria 22,24.

IMD 10 is capable of delivering anti-tachycardia pacing (ATP) therapies to heart 16. IMD 10 may detect a tachycardia within heart 16, and deliver one or more anti-tachycardia pacing (ATP) therapies to heart 16 in response to the detection. In some embodiments, IMD 10 detects a ventricular tachycardia and delivers ATP therapies via two or more leads 14 located within or proximate to ventricles 18,20, such as leads 14A and 14B shown in FIG. 1. In some embodiments, IMD 10 detects an atrial tachycardia, and delivers ATP therapies via two or more leads located within or proximate to atria 22,24.

The invention is not limited to embodiments wherein IMD 10 detects a tachycardia, however. In some embodiments, IMD 10 may receive an indication that ATP therapies should be delivered to heart 16 from another implantable or external medical device (not shown) that detects the tachycardia within heart 16. In some embodiments, IMD 10 may receive an indication that ATP therapies should be delivered from a physician, or the like, via a programmer (not shown).

Each ATP therapy delivered by IMD 10 includes one or more trains, referred to as sequences, of pacing pulses. A period between the deliveries of two consecutive pulses of a sequence is referred to as a cycle length. IMD 10 is capable of delivering pulses of a sequence of ATP pulses via one of the two or more leads 14. IMD 10 is also capable of delivering pulses of a sequence of ATP pulses via each of two or more leads 14 substantially simultaneously based on the programmed cycle lengths between consecutive pulses of the sequence. Further, as will be discussed in greater detail below, IMD 10 is capable of delivering pulses of a sequence of ATP pulses via each of two or more leads 14 at different times for each lead 14 based on programmable delay periods that are programmed for each lead 14

ATP techniques can be improved through the use of programmable delay periods and multiple sites by delivering the ATP pulses at a greater variety of locations and times. It is believed that ATP terminates a tachycardia episode through the interactions between the depolarization wave fronts caused by the ATP pulses and the depolarization wave front of the tachycardia. Delivery of ATP pulses at a greater variety of locations and times may allow these interactions to be more effective to end a particular tachycardia.

IMD 10 may also classify tachycardias. Where an ATP therapy is successful in ending a classified tachycardia, IMD 10 may associate the successful therapy and the classified tachycardia within a memory. Upon detection of a subsequent tachycardia that is similar to the classified tachycardia, the associated successful ATP therapy may be selected and delivered to heart 16. For example, if a therapy incorporating a particular set of cycle lengths between pulses and delay periods between the delivery of pulses by each electrode is successful in treating a particular tachycardia, that therapy may be selected to treat a subsequent similar tachycardia. Similarly, where an ATP therapy is not successful in ending a classified tachycardia, IMD 10 may associate the unsuccessful therapy and the classified tachycardia within the memory, and avoid selecting the unsuccessful therapy to treat a subsequent similar tachycardia. Classification of tachycardias, and selection of ATP therapies based on the success or lack of success of the therapies in treating previously classified tachycardias may further improve the effectiveness of ATP techniques.

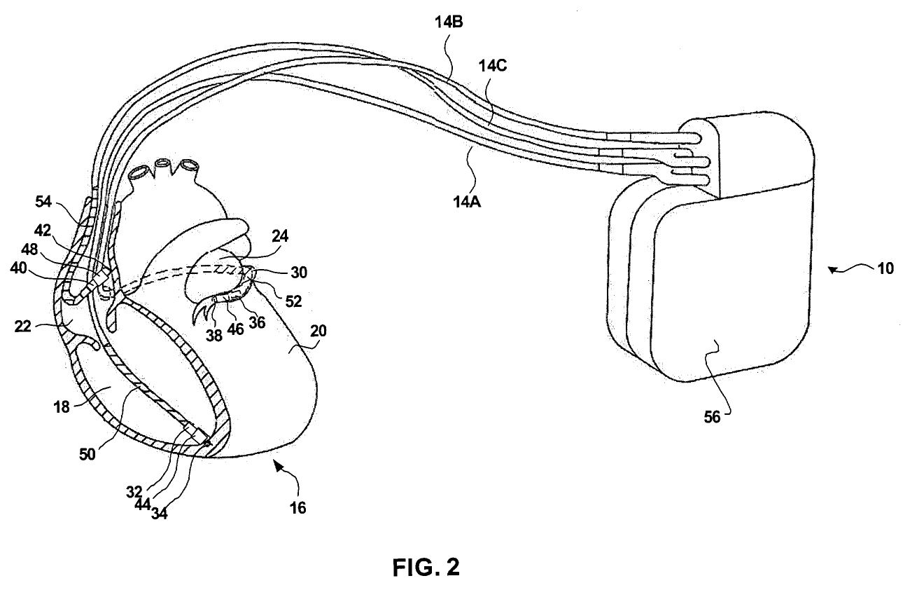

FIG. 2 is another schematic view of IMD 10 located in and near heart 16. IMD 10 may, as shown in FIG. 2, include a right ventricular (RV) lead 14A that is passed through one or more veins (not shown), the superior vena cava (not shown), and right atrium 22, and into right ventricle 18. IMD 10 may also include a left ventricular (LV) coronary sinus lead 14B that is passed through the veins, the vena cava, right atrium 22, and into the coronary sinus 38. The distal end of LV coronary sinus lead 14B is located adjacent to the wall of left ventricle 20. IMD 10 may also include additional leads 14, such as right atrial lead 14C that extends through the veins and vena cava, and into right atrium 22.

Each of leads 14 may include an elongated insulative lead body carrying a number of concentric coiled conductors separated from one another by tubular insulative sheaths. Located adjacent distal end of leads 14A, 14B and 14C are bipolar electrodes 32 and 34, 36 and 38, and 40 and 42 respectively. Electrodes 32, 36 and 40 may take the form of ring electrodes, and electrodes 34, 38 and 42 may take the form of extendable helix tip electrodes mounted retractably within insulative electrode heads 44, 46 and 48, respectively. Each of the electrodes 32-42 is coupled to one of the coiled conductors within the lead body of its associated lead 14.

Sense/pace electrodes 32, 34, 36, 38, 40 and 42 sense electrical signals attendant to the depolarization and repolarization of heart 16. The electrical signals are conducted to IMD 10 via leads 14. Sense/pace electrodes 32, 34, 36, 38, 40 and 42 further may deliver pacing and ATP pulses to cause depolarization of cardiac tissue in the vicinity thereof. The pacing and ATP pulses are generated by IMD 10 and are transmitted to sense/pace electrodes 32, 34, 36, 38, 40 and 42 via leads 14.

Leads 14A, 14B and 14C may also, as shown in FIG. 2, include elongated coil electrodes 50, 52 and 54, respectively. IMD 10 may deliver defibrillation or cardioversion shocks to heart 16 via defibrillation electrodes 50-54. Defibrillation electrodes 50-54 may be fabricated from platinum, platinum alloy or other materials known to be usable in implantable defibrillation electrodes, and may be about 5 cm in length.

The pacing system shown in FIGS. 1 and 2 is exemplary. In addition, as discussed above, the invention is not limited to the lead and electrode placements shown in FIGS. 1 and 2. In some examples, multiple electrodes are disposed for sensing and pacing multiple locations of the various heart chambers. In other words, each chamber may include a number of electrodes for sensing and pacing.

Further, the invention is not necessarily limited to the bipolar endocardial lead systems depicted in FIG. 2. Some or all of leads 14 may be epicardial leads. Further, the invention may be employed with unipolar lead systems that employ a single sense/pace electrode. Unipolar electrodes may cooperate with a remote electrode formed as part of the outer surface of the hermetically sealed housing 56 of pacemaker 10.

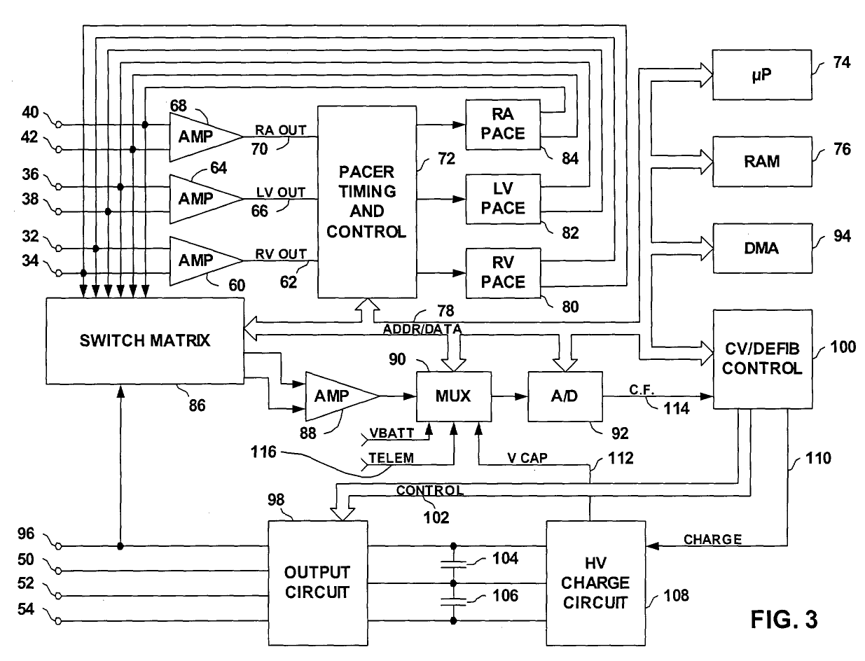

FIG. 3 is a functional block diagram of the implantable medical device of FIGS. 1 and 2. As illustrated in FIG. 3, IMD 10 may be a PCD having a microprocessor-based architecture. However, this diagram should be taken as exemplary of the type of device in which various embodiments of the present invention may be embodied, and not as limiting, as it is believed that the invention may be practiced in a wide variety of device implementations, including devices that provide ATP therapies but do not provide cardioverter and/or defibrillator functionality. The present invention is believed to find wide application to any form of IMD for use in conjunction with electrical leads.

Electrodes 32 and 34 are coupled to amplifier 60, which may take the form of an automatic gain controlled amplifier providing an adjustable sensing threshold as a function of the measured R-wave amplitude. A signal is generated on RV out line 62 whenever the signal sensed between electrodes 32 and 34 exceeds the present sensing threshold. Electrodes 36 and 38 are coupled to amplifier 64, which also may take the form of an automatic gain controlled amplifier providing an adjustable sensing threshold as a function of measured R-wave amplitude. A signal is generated on LV out line 66 whenever the signal sensed between electrodes 36 and 38 exceeds the present sensing threshold. Electrodes 40 and 42 are coupled to amplifier 68, which may take the form of an automatic gain controlled amplifier providing an adjustable sensing threshold as a function of the measured P-wave amplitude. A signal is generated on RA out line 70 whenever the signal between electrodes 40 and 42 exceeds the present sensing threshold.

Again, the configuration of sense/pace electrodes illustrated by FIGS. 1-3 is merely exemplary. IMD 10 may include any combination of two or more electrodes pairs located within or on heart 16 as discussed above with reference to FIG. 1. Depending on their location, i.e., within or on a ventricle or atrium, these electrode pairs may be coupled to either R-wave sensing circuitry, such as amplifiers 60 and 64, or P-wave sensing circuitry, such as amplifier 68.

IMD 10 may pace heart 16. Pacer timing/control circuitry 72 preferably includes programmable digital counters which control the basic time intervals associated with modes of pacing. Circuitry 72 also preferably controls escape intervals associated with pacing. In the exemplary bi-ventricular pacing environment, pacer timing/control circuitry 72 controls the ventricular escape interval that is used to time pacing pulses delivered to the ventricles.

Intervals defined by pacing circuitry 72 may also include atrial pacing escape intervals, the refractory periods during which sensed R-waves and P-waves are ineffective to restart timing of the escape intervals and the pulse widths of the pacing pulses. The durations of these intervals are determined by microprocessor 74, in response to stored data in random access memory 76 and are communicated to circuitry 72 via address/data bus 78. Pacer timing/control circuitry 72 also determines the amplitude of the cardiac pacing pulses under control of microprocessor 74.

Microprocessor 74 may operate as an interrupt driven device, and is responsive to interrupts from pacer timing/control circuitry 72 corresponding to the occurrence of sensed R-waves and corresponding to the generation of cardiac pacing pulses. Those interrupts are provided via data/address bus 78. Any necessary mathematical calculations to be performed by microprocessor 74 and any updating of the values or intervals controlled by pacer timing/control circuitry 72 take place following such interrupts.

During pacing, escape interval counters within pacer timing/control circuitry 72 may be reset upon sensing of R-waves and P-waves as indicated by signals on lines 74, 78 and 80. In accordance with the selected mode of pacing, pacer timing/control circuitry 72 triggers generation of pacing pulses by one or more of pacer output circuits 80, 82 and 84, which are coupled to electrodes 32 and 34, 36 and 38, and 40 and 42, respectively. Escape interval counters may also be reset on generation of pacing pulses and thereby control the basic timing of cardiac pacing functions.

IMD 10 may detect ventricular and/or atrial tachycardias of heart 16. Microprocessor 74 determines the durations of the intervals defined by escape interval timers via data/address bus 78. Microprocessor 74 may use the value of the count present in the escape interval counters when reset by sensed R-waves and P-waves to measure the durations of parameters such as R-R intervals, P-P intervals, P-R intervals and R-P intervals, store the measurements in memory 76, and use the measurements to detect the presence of ventricular and/or atrial tachycardias.

Detection of ventricular or atrial tachycardias, as employed in the present invention, may correspond to tachycardia detection algorithms known in the art. For example, the presence of a ventricular or atrial tachycardia may be confirmed by detecting a sustained series of short R-R or P-P intervals of an average rate indicative of tachycardia, or an unbroken series of short R-R or P-P intervals. The suddenness of onset of the detected high rates, the stability of the high rates, and a number of other factors known in the art may also be measured at this time.

IMD 10 is also capable of delivering one or more ATP therapies to heart 16. IMD 10 may detect a tachycardia and deliver one or more ATP therapies to heart 16 in response to detection, or may otherwise receive an indication that ATP therapies should be delivered, as described above. Each therapy delivered by IMD 10 includes one or more sequences of ATP pulses.

Microprocessor 74 selects a therapy from a listing of the therapies stored within a memory, such as memory 76. IMD 10 may deliver ATP therapies in a preprogrammed progression, and the order of the progression may be stored in memory 76. Microprocessor 74 may select a therapy based on a current position within the progression. Memory 76 may include program instructions that cause microprocessor 74 to detect a tachycardia, select a therapy, and direct the delivery of ATP pulses according to the selected therapy.

After microprocessor 74 selects a therapy, microprocessor 74 loads appropriate timing intervals for controlling generation of ATP pulses according to the selected therapy into pacer timing/control circuitry 72. Circuitry 72 directs one or more of output circuits 92-96 to deliver ATP pulses according to the timing intervals provided by microprocessor 74. Microprocessor 74 may determine the appropriate timing intervals based on programmed parameters for the selected ATP therapy stored in memory 76.

In order to treat a ventricular tachycardia, for example, microprocessor 74 selects an ATP therapy appropriate to treat ventricular tachycardias, i.e., an ATP therapy directed to ventricles 18 and 20 of heart 16, and, based on the stored parameters for the selected therapy, loads timing intervals into circuitry 72 which directs output circuits 92 and 94 to deliver ATP pulses to ventricles 18 and 20 according to the timing intervals. Hereinafter, the discussion of the invention will focus on the capabilities of embodiments of IMD 10 with the lead and electrode configuration illustrated in FIGS. 1-3 to deliver ATP therapies to ventricles 18 and 20 via leads 14A and 14B in response to a detection of a ventricular tachycardia. It is understood, however, that the invention encompasses embodiments of IMD 10 with a variety of lead and electrode configurations capable of treating both ventricular and atrial tachycardias.

The parameters for an ATP therapy stored in memory 76, may, for example, identify the therapy, and indicate type of ATP therapy, e.g., burst or ramp, the number of sequences within the therapy, the number of pulses within each sequence, an indication as to which electrodes are to deliver each pulse, and the cycle lengths between the various pulses of each sequence. Burst therapy provides sequences of ATP pulses wherein the cycle lengths between consecutive pulses of a sequence are the same. Ramp therapy provides sequences of ATP pulses wherein the cycle lengths between consecutive pulses decrease as pulses within the sequence are delivered. In both burst and ramp therapy, the cycle lengths and number of pulses may vary from sequence to sequence.

As mentioned above, IMD 10 is capable of delivering ATP pulses via leads 14A and 14B with a programmed delay period therebetween. Therefore, the parameters stored in memory 76 for some of the therapies include delay periods for delivery via lead 14A or lead 14B for at least some of the pulses of a sequence. In some cases, lead 14A will have a nonzero delay period, indicating that lead 14A should deliver an ATP pulse the delay period after lead 14B delivers an ATP pulse. In these cases, the delay period for lead 14B will be zero. In other cases, lead 14B will have a nonzero delay period, indicating that lead 14B should deliver an ATP pulse the delay period after lead 14A delivers an ATP pulse. In these cases, the delay period for lead 14A will be zero. In some cases, the delay period for both leads 14A and 14B may be zero, indicating that ATP pulses are to be delivered substantially simultaneously via leads 14A and 14B. The delay period may take any value, but generally nonzero delay periods will be between five and thirty milliseconds. Substantially simultaneous delivery of an ATP pulse may include delivery of the pulse via leads 14A and 14B with as much as a few second delay therebetween.

The delay period for each lead 14 may be constant within a selected therapy, but may vary from therapy to therapy. The delay periods for leads 14 may also vary from sequence to sequence within a therapy, or from ATP pulse to ATP pulse within a sequence. For example, a selected therapy may include a first sequence of burst or ramp ATP pacing with simultaneous delivery, a second sequence with a right ventricular delay period of twenty milliseconds, and a third sequence with a left ventricular delay period ten milliseconds. As another example, a selected therapy may include a sequence of burst or ramp ATP pulses where the first pulse is delivered substantially simultaneously, the second and third pulses are delivered with a right ventricular delay period of twenty milliseconds, and the fourth, fifth and sixth pulses are delivered with a left ventricular delay period ten milliseconds. A virtually unlimited variety of ATP therapies involving delay periods are possible, and the invention is not limited to any subset thereof. Based on the delay periods programmed for each electrode 14 for each ATP pulse within a selected therapy, microprocessor 74 will provide appropriate timing intervals to pacer timing/control circuit 72 such that circuitry 72 directs output circuits 80 and 82 to deliver ATP pulses at the appropriate times according to the delay periods for that pulse.

IMD 10 may also classify tachycardias. Microprocessor 74 may use digital signal analysis techniques to classify tachycardias, and to compare subsequent tachycardias with classified tachycardias. Data representing classified tachycardia may be stored in memory 76.

Switch matrix 86 is used to select which of the available electrodes are coupled to wide band (0.5-200 Hz) amplifier 88 for use in digital signal analysis. Selection of electrodes is controlled by microprocessor 74 via data/address bus 78, and the selections may be varied as desired. Signals from the electrodes selected for coupling to band pass amplifier 88 are provided to multiplexer 90, and thereafter converted to multi-bit digital signals by A/D converter 92, for storage in random access memory 76 under control of direct memory access circuit 94. Microprocessor 74 may also employ digital signal analysis techniques and characterize the digitized signals stored in random access memory 76 to recognize and classify the patient's heart rhythm and to detect ventricular or atrial fibrillation. The digital signal analysis techniques applied by microprocessor 74 may, for example, include morphology detection techniques, wavelet analysis techniques, or the measurement of R-R, P-P, R-P and/or P-R intervals, as discussed above.

Microprocessor 74 may determine whether a selected ATP therapy is successful in ending a classified tachycardia by monitoring R-R, P-P, R-P and/or P-R intervals, as discussed above, between the delivery of selected therapies, or between sequences of ATP pulses within a selected therapy. If a selected therapy is not successful, microprocessor 74 may select an additional therapy. Microprocessor may select the additional therapy by identifying the next therapy in a preprogrammed progression.

Upon delivering a selected therapy, microprocessor 74 may associate the therapy and the classified tachycardia within memory 76. Depending on whether the selected therapy was successful or unsuccessful in terminating the tachycardia, microprocessor will identify the therapy as a successful or unsuccessful therapy within memory 76. When microprocessor 74 detects subsequent tachycardias, these tachycardias may be compared to classified tachycardias. If microprocessor 74 determines that the subsequent tachycardia is similar to a classified tachycardia with an associated successful ATP therapy, microprocessor 74 may select and deliver the associated ATP therapy to treat the subsequent tachycardia. If microprocessor 74 determines that the subsequent tachycardia is similar to a classified tachycardia with one or more associated unsuccessful ATP therapies, microprocessor may select different ATP therapies to treat the subsequent tachycardia. Memory 76 may include program instructions that cause microprocessor 74 to classify tachycardias, compare tachycardias, and associate classified tachycardias with successful and unsuccessful therapies in the manner described above.

If microprocessor 74 detects a ventricular or atrial fibrillation, or if none of the ATP therapies within a preprogrammed progression was successful in terminating a ventricular or atrial tachycardia, microprocessor 74 may direct the delivery of a cardioversion or defibrillation pulse via one or more of electrodes 50, 52, 54 and 96. Electrode 96 in FIG. 3 includes the uninsulated portion of housing 56 of IMD 10. Electrodes 50, 52, 54 and 96, are coupled to high voltage output circuit 98, which includes high voltage switches controlled by CV/defib control logic 100 via control bus 102. Switches disposed within circuit 98 determine which electrodes are employed and which electrodes are coupled to the positive and negative terminals of the capacitor bank (which includes capacitors 104 and 106) during delivery of defibrillation pulses.

Microprocessor 74 may employ an escape interval counter to control timing of such cardioversion and defibrillation pulses, as well as associated refractory periods. In response to the detection of atrial or ventricular fibrillation or tachyarrhythmia requiring a cardioversion pulse, microprocessor 74 activates cardioversion/defibrillation control circuitry 100, which initiates charging of the high voltage capacitors 104 and 106 via charging circuit 108, under the control of high voltage charging control line 110. The voltage on the high voltage capacitors 104 and 106 is monitored via VCAP line 112, which is passed through multiplexer 90 and in response to reaching a predetermined value set by microprocessor 74, results in generation of a logic signal on Cap Full (CF) line 114 to terminate charging. Thereafter, timing of the delivery of the defibrillation or cardioversion pulse is controlled by pacer timing/control circuitry 72.

Delivery of cardioversion or defibrillation pulses is accomplished by output circuit 98 under the control of control circuitry 100 via control bus 102. Output circuit 98 determines whether a monophasic or biphasic pulse is delivered, the polarity of the electrodes and which electrodes are involved in delivery of the pulse. Output circuit 98 also includes high voltage switches which control whether electrodes are coupled together during delivery of the pulse. Alternatively, electrodes intended to be coupled together during the pulse may simply be permanently coupled to one another, either exterior to or interior of the device housing, and polarity may similarly be pre-set, as in current implantable defibrillators.

IMD 10 of FIG. 3 is most preferably programmable by means of an external programming unit (not shown). The programming unit may be microprocessor-based and provides a series of encoded signals to IMD 10, typically through a programming head which transmits or telemeters radio-frequency (RF) encoded signals to IMD 10. Microprocessor 74 may receive these signals via antenna 116, multiplexer 90, A/D converter 92 and address/data bus 78. A user, such as a physician or clinician, can program IMD 10 via the programmer. The user may, for example, program parameters of ATP therapies, specify a programmed progression of therapies, or direct IMD 10 to deliver ATP therapies via the programmer.

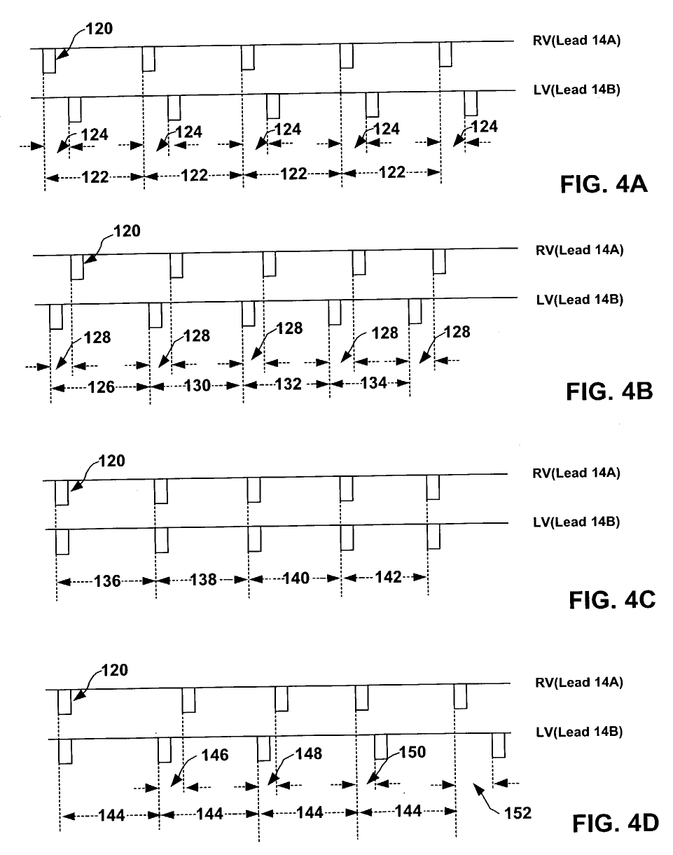

FIGS. 4A-D are timing diagrams illustrating the delivery of ATP pulses 120 by IMD 10 according to the invention. For ease of illustration, only a single ATP pulse 120 is labeled in each of FIGS. 4A-D. Each of FIGS. 4A-D depict a five-pulse sequence of ATP pulses. However, sequences of ATP pulses may include any number of pulses. The sequences depicted together in FIGS. 4A-D may form a single ATP therapy, or each sequence may be a part of a separate ATP therapy. Moreover, the invention is not limited to the sequences depicted. As mentioned above, a virtually unlimited variety of ATP therapies according to the invention are possible, and the invention is not limited to any subset thereof. For example, although each sequence illustrated in FIGS. 4A-D includes delivery of each pulse by both leads 14, sequences delivered consistent with the invention may include delivery of some of the pulses via a single lead 14 based on the programmed cycle lengths between those pulses and previous pulses within the sequence.

As discussed above, after microprocessor 74 selects a therapy, microprocessor 74 loads appropriate timing intervals for controlling generation of ATP pulses according to the selected therapy into pacer timing/control circuitry 72 based on the stored parameters for the selected therapy. The parameters for an ATP therapy stored in memory 76, may, for example, identify the therapy, and indicate type of ATP therapy, e.g., burst or ramp, the number of sequences within the therapy, the number of pulses within each sequence, an indication as to which electrodes are to deliver each pulse, cycle lengths between the various pulses of each sequence, and delay periods for delivery via lead 14A and lead 14B for at least some of the pulses. Circuitry 72 directs output circuits 92 and 94 to deliver ATP pulses to ventricles 18 and 20 according to the timing intervals. As discussed above, the delay period may for each lead 14 may be constant within a selected therapy, but may vary from therapy to therapy, may vary from sequence to sequence within a therapy, or from ATP pulse to ATP pulse within a sequence.

FIG. 4A illustrates an exemplary burst sequence of ATP pulses 120 with a constant cycle length 122. As can be seen in FIG. 4A, delivery of ATP pulses 120 to left ventricle 20 via lead 14B is delayed in comparison to delivery of ATP pulses 120 to the right ventricle 18 via lead 14A by a delay period 124. The parameters for this sequence may indicate the that the type is burst, that the number of pulses 120 is five, the cycle length 122 for each pulse 120, and that a delay period 124 applies to lead 14B for each pulse 120. Based on these parameters, microprocessor 74 will provide timing intervals to circuitry 72, which will direct output circuit 80 to deliver a pulse 120 via lead 14A, and then a pulse 120 via lead 14A each cycle length 122 thereafter, and direct output circuit 82 to deliver a pulse 120 via lead 14B the delay period 124 after each time directing output circuit 80 to deliver a pulse 120.

FIG. 4B illustrates an exemplary ramp sequence of ATP pulses 120 where the cycle lengths 126, and 130-134 become shorter as the sequence progresses. As can be seen in FIG. 4B, delivery of ATP pulses 120 to right ventricle 18 via lead 14A is delayed in comparison to delivery of ATP pulses 120 to the left ventricle 20 via lead 14B by a delay period 128. The parameters for this sequence may indicate the that the type is ramp, that the number of pulses 120 is five, the cycle length 126,130-134 for each pulse, and that a delay period 128 applies to lead 14A for each pulse. Based on these parameters, microprocessor 74 will provide timing intervals to circuitry 72, which will direct output circuit 82 to deliver pulses 120 via lead 14B according to the cycle lengths 126,130-134, and direct output circuit 80 to deliver a pulse 120 via lead 14A the delay period 124 after each time directing output circuit 82 to deliver a pulse 120 via lead 14B.

FIG. 4C illustrates another exemplary ramp sequence of ATP pulses 120 where the cycle lengths 135-142 become shorter as the sequence progresses. As can be seen in FIG. 4C, deliver of ATP pulses 120 via leads 14A and 14B is substantially simultaneous. As mentioned above, substantially simultaneous delivery includes delivery via leads 14A and 14B that is separated by as much as a few milliseconds. The parameters for this sequence may indicate that the type is a ramp, that the number of pulses 120 is five, the cycle length 136-142 for each pulse, and that the delivery by leads 14A and 14B is to be substantially simultaneous, e.g., that no delay period applies to either of leads 14A and 14B, or that the delay period for both leads 14A and 14B is zero. Based on these parameters, microprocessor 74 will provide timing intervals to circuitry 72, which will direct output circuits 80 and 82 to deliver pulses 120 via leads 14A and 14B substantially simultaneously according to the cycle lengths 136-142.

FIG. 4D illustrates another exemplary burst sequence of ATP pulses 120 delivered via leads 14A and 14B. The parameters for this sequence may indicate that the type is burst, that the number of pulses 120 is five, the cycle length 144 for each pulse 120, and the delay period for each of leads 14A and 14B for each pulse 120. Based on these parameters, pacer timing/control circuitry 72 directs output circuit 80 and 82 to deliver a first pulse 120 of the sequence via leads 14A and 14B at substantially the same time, e.g., the delay period for each of leads 14A and 14B for the first pulse 120 is zero. A cycle length 144 after delivery of the first pulse 120 via leads 14A and 14B, circuitry 72 directs output circuit 82 to deliver the second pulse 120 of the sequence via lead 14B, e.g., the delay period for lead 14B for the second pulse 120 is zero. There is a nonzero delay period 146 for lead 14A for the second pulse 120, thus circuitry 72 will direct output circuit 80 to deliver the second pulse 120 via lead 14A the delay period 146 after directing output circuit 82 to deliver of the second pulse 120 via lead 14B. A cycle length 144 after delivery of the second pulse 120 via lead 14B, circuitry 72 directs output circuit 82 to deliver the third pulse 120 of the sequence via lead 14B, e.g., the delay period for lead 14B for the third pulse is zero. There is a nonzero delay period 148 for lead 14A for the third pulse 120 of the sequence, thus circuitry 72 will direct output circuit 80 to deliver the third pulse 120 via lead 14A the delay period 148 after directing output circuit 82 to deliver of the third pulse 120 via lead 14B.

A cycle length 144 after delivery of the third pulse 120 via lead 14B, circuitry 72 directs output circuit 80 to deliver the fourth pulse 120 of the sequence via lead 14A, e.g., the delay period for lead 14A for the fourth pulse is zero. There is a nonzero delay period 150 for lead 14B for the fourth pulse, thus circuitry 72 will direct output circuit 82 to deliver the fourth pulse 120 via lead 14B the delay period 150 after directing output circuit 80 to deliver of the fourth pulse 120 via lead 14A. A cycle length 144 after delivery of the fourth pulse 120 via lead 14A, circuitry 72 directs output circuit 80 to deliver the fifth pulse 120 of the sequence via lead 14A, e.g., the delay period for lead 14A for the fifth pulse 120 is zero. There is a nonzero delay period 152 for lead 14B for the fifth pulse 120, thus circuitry 72 will direct output circuit 82 to deliver the fifth pulse 120 via lead 14B the delay period 152 after directing output circuit 80 to deliver of the fifth pulse 120 via lead 14A.

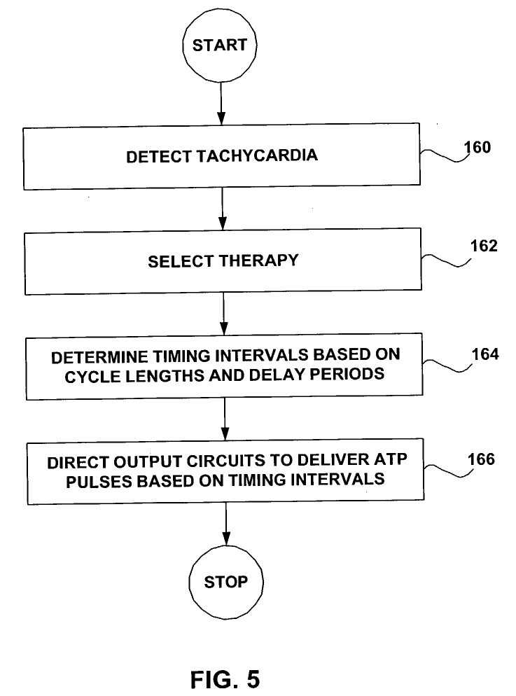

FIG. 5 is a flow chart illustrating an exemplary method for delivery of anti-tachycardia pacing therapy by a medical device, such as IMD 10 or an external pacing system. For purposes of example, the method is described in reference to IMD 10.

Initially, IMD 10 may detect a tachycardia within heart 16 (160), and select a therapy in response to the detection (162) by any of the methods described above. For example, a microprocessor 74 of IMD 10 may detect a tachycardia based R-R intervals P-P intervals, R-P intervals and P-R intervals determined based on values of counters maintained by pacer timing/control circuitry 72 when reset by detection of R-waves or P-waves or delivery of a pacing pulse, as described above. Microprocessor 74 may select a therapy from preprogrammed progression of therapies, or based on a comparison to a classified tachycardia with an associated successful therapy, as described above.

IMD 10 then determines timing intervals for the delivery of each of the ATP pulses of the selected therapy via each of two or more leads 14 based on stored parameters for the selected therapy (164), and delivers ATP pulses via each of the two or more leads 14 according to the timing intervals for each lead 14 (166). Depending on the leads included with IMD 10 and the type of tachycardia detected, i.e., ventricular or atrial, IMD 10 may select the two or more leads 14 for delivery of ATP pulses from a plurality of leads 14. A microprocessor 74 of IMD 10 determines the timing intervals for each lead 14 based on the programmed cycle lengths between consecutive pulses and delay periods that are programmed for each lead for each ATP pulse. The microprocessor 74 may provide the timing intervals to circuitry, such as pacer timing/control circuitry 72, that directs output circuits for each lead 14, such as output circuits 80 and 82 for leads 14A and 14B, to deliver pacing pulses via each lead 14 according to the timing intervals.

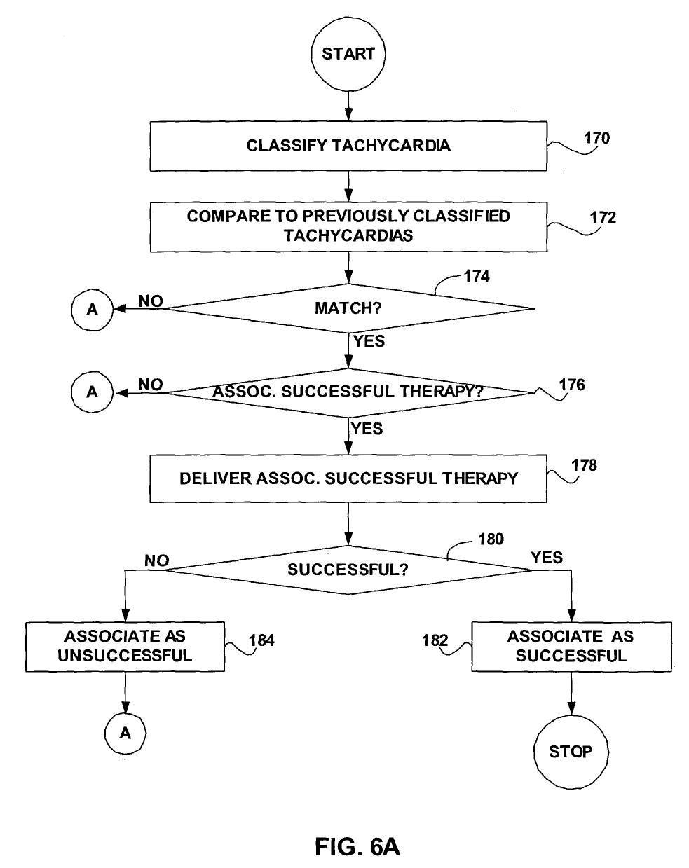

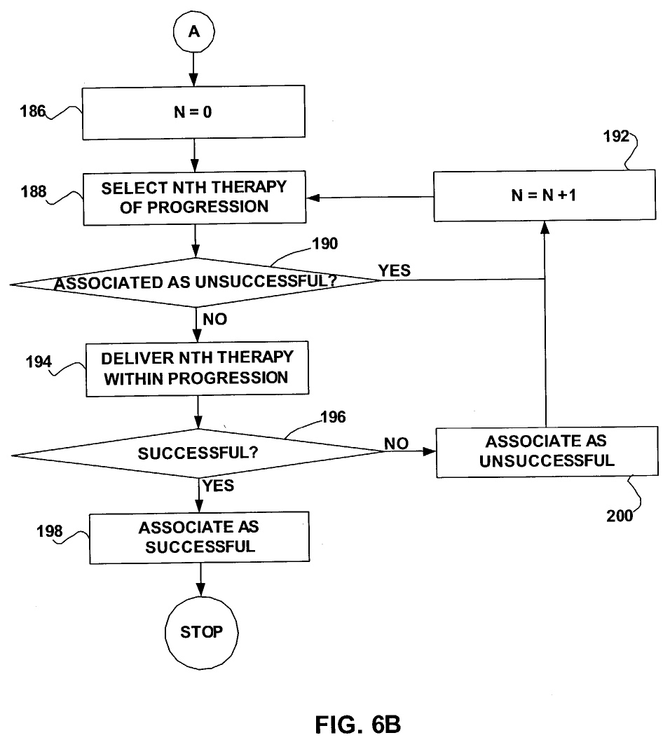

FIGS. 6A and 6B are flow charts illustrating an exemplary method for classifying tachycardias and selecting anti-tachycardia pacing therapies that may be preformed by a medical device, such as IMD 10, or an external pacing system. For purposes of example, the method is described in reference to IMD 10.

IMD 10 classifies a tachycardia using any of the methods described above (170), such as a digital signal analysis of electrical activity within heart 16 and morphology detection by a microprocessor 74 of the IMD 10. The microprocessor 74 may compare the newly classified tachycardia to data stored in memory 76 representative of previously classified tachycardias (172). If microprocessor 74 determines that the newly classified tachycardia matches a previously classified tachycardia (174), e.g., is sufficiently similar to the previously classified tachycardia according to some criterion such as a threshold, microprocessor 74 will determine whether the previously classified tachycardia is associated with a successful therapy within memory 76 (176). If the previously classified tachycardia is associated with a successful therapy, microprocessor 74 may direct the delivery of the associated successful therapy (178), determine whether the associated successful therapy was successful in ending the newly classified tachycardia (180), and, if successful, associate the therapy with the newly classified tachycardia in memory 76 as a successful therapy (182). If microprocessor 74 determines that the therapy associated with the previously classified tachycardia was not successful in ending the newly classified tachycardia, microprocessor 74 may associate the therapy with the newly classified tachycardia as an unsuccessful therapy (184). Microprocessor 74 may determine whether a selected therapy is successful in ending a classified tachycardia by monitoring R-R, P-P, R-P and/or P-R intervals, as discussed above, after delivery of the therapy, or between sequences of ATP pulses within the therapy.

If microprocessor 74 determines that the newly classified tachycardia does not match any previously classified tachycardia (174), determines that the previously classified tachycardia is not associated with a successful therapy (176), or determines that delivery of an associated successful therapy was not successful in terminating the tachycardia (180), microprocessor 74 will select and cause the delivery of one or more therapies within a preprogrammed progression of therapies (186-200) that may be stored in memory 76 as described above. The microprocessor 74 may determine whether the each selected therapy of the progression has been previously associated with either the newly classified tachycardia or a similar previously identified tachycardia as an unsuccessful therapy (190). If a selected therapy within the progression has been previously associated as an unsuccessful therapy, microprocessor 74 may select the next therapy in the progression (192,188). If a selected therapy within the progression has not been previously associated as an unsuccessful therapy, microprocessor 74 may deliver the selected therapy (194), determine whether the selected therapy was successful in terminating the newly classified tachycardia (196), and associate the selected therapy with the newly classified tachycardia as a successful or unsuccessful therapy based on the determination (198,200). If the selected therapy from the progression is not successful in terminating the newly classified tachycardia, processor 74 may select the next therapy in the progression (192,188). If the preprogrammed progression of ATP therapies is exhausted without terminating the newly detected tachycardia, microprocessor 74 may deliver the therapies within the progression that were passed over because they were associated with a similar previously classified tachycardia as unsuccessful, select a new progression of therapies, or deliver a cardioversion or defibrillation pulse.

Various embodiments of the invention have been described. It is to be understood, however, that in light of this disclosure, other embodiments will become apparent to those skilled in the art. The techniques described herein may be embodied in methods, or implantable medical devices that carry out the methods. For example, a medical device may include a number of electrodes coupled to a control unit via implantable leads. The control unit may include components that perform the functions ascribed to components described herein, such as pacer timing/control circuit 72 and microprocessor 74. The implantable medical device may include two or more electrodes configured in any manner consistent with the disclosure. Some embodiments may be practiced in an external (non-implantable) or a partially external pacemaker device. In other embodiments, the invention may be directed to a computer readable medium comprising program code that causes an external or implantable medical device such as a pacemaker to carry out methods in accordance with the invention. In that case, the medium may store computer readable instructions, and the external or implantable medical device may include a processor that executes the instructions in order to perform the methods. Accordingly, these and other embodiments are within the scope of the following claims.

What is claimed is:

1. A method comprising:
selecting an anti-tachycardia pacing therapy that includes at least one sequence of pulses; and
delivering at least some of the pulses of at least one sequence to a heart via each of at least two electrodes based on programmed cycle lengths between consecutive pulses of the sequence and delay periods that are programmed for each of the electrodes.

2. The method of claim 1, wherein delivering the pulses comprises:
delivering a pulse within the sequence to the heart via a first electrode at a first time based on the programmed cycle length between the pulse and a previous pulse within the sequence; and
delivering the pulse to the heart via a second electrode at a second time that is the delay period programmed for the second electrode for the pulse after the first time.

3. The method of claim 2, wherein the delay period programmed for the first electrode is zero, and the delay period programmed for the second electrode is a nonzero value.

4. The method of claim 1, wherein the delay periods programmed for each of the electrodes for a pulse within the sequence are equal, and delivering the pulses comprises delivering the pulse via each of the electrodes at substantially the same time based on the programmed cycle length between the pulse and a previous pulse within the sequence.

5. The method of claim 4, wherein the delay periods are zero.

6. The method of claim 1, wherein delivering at least some of the pulses comprises delivering some of the pulses of the sequence via a single electrode based on the programmed cycle lengths.

7. The method of claim 1, wherein delivering the pulses comprises:
delivering a first sequence of pulses via each of the electrodes based on a first set of programmed delay periods that apply to each pulse in the first sequence; and
delivering a second sequence of pulses via each of the electrodes based on a second set of programmed delay periods that apply to each pulse in the second sequence.

8. The method of claim 1, wherein delivering the pulses comprises:
delivering a first pulse within the sequence via each of the electrodes based on a first set of programmed delay periods; and
delivering a second pulse within the sequence via each of the electrodes based on a second set of programmed delay periods.

9. The method of claim 1, further comprising storing parameters for the therapy, wherein the parameters include the programmed cycle lengths and the programmed delay periods.

10. The method of claim 1, further comprising receiving parameters for the therapy via a programmer, wherein the parameters include the programmed cycle lengths and programmed delay periods.

11. The method of claim 1, further comprising:
detecting a tachycardia of a heart; and
selecting the anti-tachycardia pacing therapy in response to the detection.

12. The method of claim 11, wherein detecting a tachycardia comprises detecting a ventricular tachycardia, and delivering the pulses comprises delivering each of the pulses via at least two electrodes located at least one of proximate and within ventricles of the heart.

13. The method of claim 11, wherein detecting a tachycardia comprises detecting an atrial tachycardia, and delivering the pulses comprises delivering each of the pulses via at least two electrodes located at least one of proximate and within atria of the heart.

14. The method of claim 11, further comprising:
classifying the detected tachycardia;
storing data representing the classified tachycardia in a memory;
determining whether the selected therapy was successful in ending the detected tachycardia; and
associating the selected therapy with the classified tachycardia within the memory based on the determination.

15. The method of claim 11, wherein selecting a therapy comprises:
determining whether the detected tachycardia is similar to a previously classified tachycardia; and
selecting a therapy associated with the previously classified tachycardia based on the determination.

16. The method of claim 11, wherein detecting a tachycardia comprises detecting the tachycardia with one of an implantable medical device and an external medical device.

17. The method of claim 1, further comprising storing a progression of therapies, wherein selecting a therapy comprises selecting a therapy from the progression based on a current position in the progression.

18. The method of claim 1, wherein selecting a therapy comprises selecting the therapy in response to commands received from a programmer.

19. The method of claim 1, wherein delivering the pulses comprises delivering the pulses with one of an implantable medical device and an external medical device.

20. The method of claim 1, wherein delivering the pulses comprises delivering the pulses via each of at least two bipolar electrode pairs.

21. A device comprising:
at least two electrodes to deliver pacing pulses to a heart; and
a control unit to select an anti-tachycardia pacing therapy that includes at least one sequence of pulses, and direct output circuits associated with the electrodes to deliver at least some of the pulses of at least one sequence to the heart via each of the electrodes based on programmed cycle lengths between consecutive pulses of the sequence and delay periods that are programmed for each of the electrodes.

22. The device of claim 21, wherein the control unit directs a first output circuit to deliver a pulse within the sequence to the heart via a first electrode at a first time based on the programmed cycle length between the pulse and previous pulse within the sequence, and directs a second output circuit to deliver the pulse to the heart via a second electrode at a second time that is the delay period programmed for the second electrode for the pulse after the first time.

23. The device of claim 21, wherein the delay periods programmed for each of the electrodes for a pulse within the sequence are equal, and the control unit directs output circuits associated with the electrodes to deliver the pulse via each electrode at substantially the same time based on the programmed cycle between the pulse and a previous pulse within the sequence.

24. The device of claim 21, wherein the control unit directs one of the output circuits to direct some of the pulses of the sequence via a single electrode based on the programmed cycle lengths.

25. The device of claim 21, wherein the control unit directs the output circuits to deliver a first sequence of pulses via each of the electrodes based on a first set of programmed delay periods that apply to each pulse in the first sequence, and directs the output circuits to deliver a second sequence of pulses via the electrodes based on second set of programmed delay periods that apply to each pulse in the second sequence.

26. The device of claim 21, wherein the control unit directs the output circuits to deliver a first pulse within the sequence via each of the electrodes based on a first set of programmed delay periods, and directs the output circuits a second pulse within the sequence via the electrodes based on a second set of programmed delay periods.

27. The device of claim 21, further comprising a memory to store parameters for the therapy, wherein the parameters include the programmed cycle lengths and programmed delay periods.

28. The device of claim 21, further comprising a telemetry antenna, wherein the control unit receives parameters for the therapy via a programmer and the antenna, and the parameters include the programmed cycle lengths and programmed delay periods.

29. The device of claim 21, wherein the electrodes sense electrical activity within the heart, and the control unit detects a tachycardia of the heart based on the electrical activity, and selects the therapy based on the detection.

30. The device of claim 29, wherein the control unit detects a ventricular tachycardia, and directs output circuits associated with at least two electrodes located at least one of proximate and within ventricles of the heart to deliver each of the pulses.

31. The device of claim 29, wherein the control unit detects an atrial tachycardia, and directs output circuits associated with at least two electrodes located at least one of proximate and within atria of the heart to deliver each of the pulses.

32. The device of claim 29, further comprising a memory, wherein the control unit classifies the detected tachycardia based on the electrical activity sensed via the electrodes, stores data representing the classified tachycardia in the memory, determines whether the selected therapy was successful in ending the detected tachycardia based on the sensed electrical activity, and associates the selected therapy with the classified tachycardia within the memory based on the determination.

33. The device of claim 29, wherein the control unit selects a therapy by determining whether the detected tachycardia is similar to a previously classified tachycardia, and selecting a therapy associated with the previously classified tachycardia based on the determination.

34. The device of claim 21, further comprising a memory to store a progression of therapies, wherein the control unit selects a therapy by selecting a therapy from the progression based on a current position in the progression.

35. The device of claim 21, further comprising a telemetry antenna, wherein the control unit selects the therapy in response to commands received from another medical device via the antenna.

36. The device of claim 35, wherein the other medical device is a programmer.

37. The device of claim 21, wherein the device is implanted within a patient.

38. The device of claim 21, wherein the electrodes comprise bipolar electrode pairs.

39. The device of claim 21, wherein the control unit comprises a microprocessor.

40. A computer-readable medium comprising instructions that cause a programmable processor to:
select an anti-tachycardia pacing therapy that includes at least one sequence of pulses; and
deliver at least some of the pulses of at least one sequence to the heart via each of at least two electrodes based on programmed cycle lengths between consecutive pulses of the sequence and delay periods that are programmed for each of the electrodes.

41. The computer-readable medium of claim 40, wherein the instructions that cause a processor to deliver the pulses comprise instructions that cause the processor to:
deliver a pulse within the sequence to the heart via a first electrode at a first time based on the programmed cycle length between the pulse and a previous pulse within the sequence; and
deliver the pulse to the heart via a second electrode at a second time that is the delay period programmed for the second electrode for the pulse after the first time.

42. The computer-readable medium of claim 40, wherein the delay periods programmed for each the electrodes for a pulse are equal, and the instructions that cause a processor to deliver the pulses comprises instructions that cause a processor to deliver the pulse via each of the electrodes at substantially the same time based on the programmed cycle length between the pulse and a previous pulse within the sequence.

43. The computer-readable medium of claim 40, further comprising instructions that cause a processor to detect a tachycardia of a heart, wherein the instructions that cause a processor to select a therapy comprise instructions that cause a processor to select a therapy based on the detection.

44. The computer-readable medium of claim 43, further comprising instructions that cause a processor to:
classify the detected tachycardia;
store data representing the classified tachycardia in a memory;
determine whether the selected therapy was successful in ending the detected tachycardia; and
associate the selected therapy with the classified tachycardia within the memory based on the determination.

45. The computer-readable medium of claim 43, wherein the instructions that cause a processor to select a therapy comprise instructions that cause a processor to:
determine whether the detected tachycardia is similar to a previously classified tachycardia; and
select a therapy associated with the previously classified tachycardia based on the determination.

46. The computer-readable medium of claim 40, further comprising instructions that cause a processor to store a progression of therapies, wherein the instructions that cause a processor to select a therapy comprise instructions that cause a processor to select a therapy from the progression based on a current position in the progression.

47. A method comprising:
detecting a tachycardia of a heart with a medical device;
automatically selecting an anti-tachycardia pacing therapy that includes at least one sequence of pulses in response to the detection;
delivering a pulse within the sequence to the heart via a first electrode at a first time; and
delivering the pulse to the heart via a second electrode at a second time that is subsequent to the first time.

48. The method of claim 47, wherein the second time is a programmed delay period associated with the second electrode subsequent to the first time.
